(19) United States
(12) Patent Application Publication
(10) Pub. No.: US 2024/0366194 A1
(43) Pub. Date: Nov. 7, 2024

(54) DEVICES AND METHODS FOR MICROBIOME SAMPLING

(71) Applicant: AOBIOME LLC, Cambridge, MA (US)

(72) Inventors: Lauren Nicole Ambrogio, Boulder, CO (US); Larry Weiss, San Francisco, CA (US)

(21) Appl. No.: 18/776,066

(22) Filed: Jul. 17, 2024

Related U.S. Application Data
(63) Continuation of application No. 18/400,681, filed on Dec. 29, 2023.
Application No. 18/400,681 is a continuation of application No. 16/956,930, filed on Jun. 22, 2020, now abandoned.
(60) Provisional application No. 62/609,767, filed on Dec. 22, 2017.

(51) Int. Cl. A61B 10/00; A61F 13/38; C12Q 1/6888; G01N 33/569

(52) U.S. Cl. CPC A61B 10/0045; A61B 10/0038; A61B 10/0051; A61B 10/0064; A61F 13/38; C12Q 1/6888; G01N 33/569; A61B 2010/0054; A61B 2010/0067

(57) ABSTRACT

Devices and methods for microbiome sampling and evaluation are disclosed. Devices including a capture substrate having first and second regions suitable for first and second analytic methods, respectively, are disclosed. Methods for collecting the microbiome sample with such devices are disclosed. Methods of evaluating the microbiome sample collected with such devices are disclosed. Kits including the devices for sampling and evaluating microbiome samples are also disclosed.

### CROSS-REFERENCE TO RELATED APPLICATIONS

This application is a continuation of U.S. patent application Ser. No. 18/400,681, filed on Dec. 29, 2023, which is a continuation of U.S. patent application Ser. No. 16/956,930, filed on Jun. 22, 2020, which is a U.S. national phase application, and claims the benefit of priority under 35 U.S.C. § 371, of International (PCT) Patent Application Serial No. PCT/US2018/067208, filed on Dec. 21, 2018, which claims priority under 35 U.S.C. § 119 (e) to U.S. Provisional Patent Application Ser. No. 62/609,767, filed on Dec. 22, 2017, the entire disclosure of each of which is hereby incorporated herein by reference in its entirety for all purposes.

### FIELD OF THE TECHNOLOGY

Aspects relate generally to the microbiome and, more specifically, to the collection of a sample of the microbiome.

### BACKGROUND

Bacteria and other microorganisms are ubiquitous in the environment. The discovery of pathogenic bacteria and the germ theory of disease have had a tremendous effect on health and disease states. Microorganisms are a normal part of the environment of all living things and may be beneficial. In the gut, for example, bacteria are not pathogenic under normal conditions, and in fact improve health by rendering the normal intestinal contents less hospitable for disease causing organisms.

### SUMMARY

In accordance with one or more aspects, a device for obtaining a microbiome sample from a subject is disclosed. The device may comprise a capture substrate, wherein the capture substrate is configured to contain: a first region suitable for a first analytic method, and a second region suitable for a second analytic method. The first region and the second region may be configured to obtain microbiome sample from the subject.

In accordance with one or more aspects, a device for obtaining a microbiome sample from a subject is disclosed. The device may comprise a roller element having an outer surface and a transverse axis, the roller element configured to rotate about its transverse axis, and a capture substrate disposed on the outer surface and configured to allow collection of a microbiome sample. The capture substrate may be suitable for a first microbiome analytic method.

In some aspects, the first and second regions may obtain the same or substantially the same microbiome sample from the subject. The device may be configured such that upon movement across a surface perpendicular to the transverse axis, a first portion of the capture substrate is brought into contact with the subject, and upon further movement across the surface perpendicular to the transverse axis a second portion of the capture substrate is brought into contact with the subject. In at least some aspects, prior to contact with the subject: (i) the capture substrate is substantially free of a preselected member of the microbiome, e.g. an AOM; (ii) the capture substrate has less than a threshold percentage of a preselected member of the microbiome, e.g., an AOM; or (iii) the capture substrate has a predetermined level of a preselected member of the microbiome, e.g., an AOM.

In some aspects, the device may comprise a plurality of segments of capture substrate. The device may be configured such that after collection, a first loaded or used segment can be removed. The device may be configured such that after removal of a first loaded or used segment, a second segment is available for use. The capture substrate may comprise a predetermined surface area. The device may be configured to apply a predetermined amount of pressure to a surface of the subject. In some embodiments, the capture substrate may comprise a reagent suitable for the first analytic method. In at least some aspects, the capture substrate may be configured to contain: a first region comprising a first reagent suitable for a first analytic method; and a second region comprising a second reagent suitable for a second analytic method. The first region and the second region may be configured to capture microbiome sample from a subject.

In some aspects, the device may further comprise a handle. The handle may be configured to be separable into a first handle portion associated with the first region of capture substrate and a second handle portion associated with the second region of capture substrate. The handle may be configured to be separated from the capture substrate. The first handle portion may be separable from the second handle portion. The first handle portion may be separable from the first region of capture substrate, and the second handle portion may be separable from the second region of capture substrate. The first region may be disposed on a first surface, and the second region may be disposed on a second surface. The first region and the second region may alternatively be disposed on the same surface. The surface may be configured, e.g., with a groove, indent, or perforation, to promote separation of the first region from the second region.

In some aspects, the first and second regions may sample microbiome simultaneously. The device may further comprise a second roller element. The capture substrate or region thereof may comprise a swab or flock. The capture substrate may be configured to be received by a receptacle associated with a microbiome analytic method.

In accordance with one or more aspects, a method of collecting a microbiome sample from a subject is disclosed. The method may comprise contacting the subject with a capture substrate, wherein the capture substrate is configured to contain: a first region suitable for a first analytic method; and a second region suitable for a second analytic method. The first region and the second region may be configured to sample microbiome from the subject. The contacting may be sufficient to transfer a microbiome sample to the capture substrate, thereby providing a microbiome sample from a subject.

In accordance with one or more aspects, a method of evaluating a microbiome sample from a subject is disclosed. The method may comprise providing a capture substrate configured to contain: a first region suitable for a first analytic method; and a second region suitable for a second analytic method. The first region and the second region may be configured to capture microbiome sample from the subject, and the capture substrate may have been contacted with the subject. The method may further comprise forming one or both of: (i) a first reaction mixture comprising microbiome sample from the first region; and (ii) a second reaction mixture comprising microbiome sample from the second region.

In some aspects, the contacting may be sufficient to transfer microbiome sample from the subject to the first and second regions of the capture substrate. The contacting may be sufficient to obtain the same or substantially the same microbiome sample from the subject on the first and second regions of the capture substrate. The contacting may be sufficient to transfer microbiome sample from the subject to the first and second regions of the capture substrate substantially simultaneously.

The method may further comprise separating the first region from the second region. The first region and/or the second region may be contacted with the face, neck, scalp, head, shoulder, arm, leg, underarm, torso, chest, feet, knee, ankle, or buttocks of the subject.

In accordance with certain embodiments, the capture substrate provided may be configured to contain: a first region comprising a first reagent suitable for a first analytic method, and a second region comprising a second reagent suitable for a second analytic method. The first region and the second region may be configured to capture a microbiome sample from a subject. The first region and the second region may be configured to capture the same or substantially the same microbiome sample from the subject. The first region and the second region may be configured to obtain the microbiome sample substantially simultaneously.

The method may further comprise forming a first reaction mixture comprising microbiome sample from the first region. The method may further comprise forming a second reaction mixture comprising microbiome sample from the second region. The method may further comprise providing a value for a parameter related to the microbiome sample on the first region. The method may further comprise providing a value for a parameter related to a first analytic method, e.g., a nucleic acid sequence, e.g., DNA sequence or RNA sequence. The method may further comprise providing a value for a parameter related to the microbiome sample on the second region. The method may further comprise providing a value for a parameter related to the second analytic method e.g., a microbial colony forming unit (CFU) concentration.

In some aspects, the first analytic method comprises a manipulation of a nucleic acid. The first analytic method may comprise isolation or purification of a nucleic acid. The first analytic method may comprise sequencing a nucleic acid, e.g., DNA or RNA. The first analytic method may comprise sequencing a nucleic acid with a NGS method. The first analytic method may comprise contacting a nucleic acid with a restriction fragment. The second analytic method may comprise culturing the microbiome sample. The second analytic method may comprise obtaining a snapshot of the microbiome sample. The first or second analytic method may involve preserving, e.g., freezing the microbiome sample.

In some aspects, the capture substrate or region thereof may comprise a roller. The capture substrate or region thereof may comprise a swab or flock. The method may further comprise transmitting the value for a parameter related to the microbiome sample on the first and/or second region to a database. The method may further comprise correlating the value for a parameter related to the microbiome sample on the first and/or second region. The method may further comprise modulating a microbiome of the subject based on the value for a parameter related to the microbiome sample on the first and/or second region.

In some aspects, the method may further comprise separating a handle associated with the capture substrate. The method may further comprise separating a handle from the capture substrate. The method may further comprise contacting the microbiome sample captured on the first region to a first reagent suitable for the first analytic method and contacting the microbiome sample captured on the second region to a second reagent suitable for the second analytic method. The method may further comprise introducing the capture substrate or region thereof to a buffer solution. The method may further comprise contacting the capture substrate with the skin, face, neck, scalp, head, shoulder, arm, leg, underarm, torso, chest, feet, knee, ankle, buttocks, anus, oral cavity, nasal cavity, eye, or ear of the subject.

In accordance with one or more aspects, a kit for evaluating a microbiome sample from a subject is disclosed. The kit may comprise a collection device as described herein.

In some aspects, the kit may further comprise instructions for performing any of the methods of providing a microbiome sample as described herein. The kit may further comprise instructions for performing any of the methods of evaluating a microbiome sample as described herein. The kit may further comprise a container for handling the capture substrate or region thereof. The kit may further comprise a source of one or more buffer solutions. In at least some aspects, the kit and/or component(s) thereof may be substantially sterile. The kit and/or component(s) thereof may be substantially free of preservatives. In some aspects, the buffer solution may comprise a preservative. The kit may further comprise a source of a protein or a lipid. In at least some aspects, the kit may be barcoded. Likewise, at least one component of the kit may be barcoded.

In accordance with one or more aspects, a database comprising values for one or more parameters related to one or more microbiome analytic methods as described herein is disclosed.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A and 1B provide a schematic of a microbiome sampling device including a swab in accordance with one or more embodiments; and

FIGS. 2A and 2B provide a schematic of a microbiome sampling device including a roller element in accordance with one or more embodiments.

### DETAILED DESCRIPTION

In accordance with one or more embodiments, a microbiome of a subject may be sampled. Various regions of a subject may be associated with unique microbiomes. For example, the skin of a subject may have a distinct microbiome from the gut, nasal cavity, lung, or anus of the subject. Likewise, the microbiome of the skin, for example, may itself be localized and vary across a surface of a subject. Each microbiome may involve a discrete community of bacteria, yeasts, parasites, and other microorganisms. Some of these microorganisms may be nonpathogenic and others may be pathogenic, e.g., some may be beneficial and others unbeneficial. Without wishing to be bound by any particular theory, it may be desirable to evaluate and/or preserve a microbiome sample from a target area of a subject. Beneficially, a single microbiome sample may be obtained and subjected to one, two, or more analytical methods in accordance with various embodiments to facilitate precise analysis. The same or substantially the same microbiome sample may be obtained at one, two, or more regions, e.g. of capture substrate, associated with a collection device and each region may then beneficially undergo different processing.

In accordance with one or more embodiments, devices and methods may be used to sample a microbiome of a subject. The sample may then be divided for present and/or future use in various analytical techniques. The same or substantially the same microbiome sample may be captured at different portions of capture substrate and used or saved for different purposes. As noted above, a microbiome sample of a subject may include a community of various microorganisms as commonly known to those of skill in the relevant art. The devices and methods disclosed herein may be used to generate a profile of a microbiome sample. For example, a community of microorganisms may be identified. The ratio or prevalence of such microorganisms within the community may be evaluated, for example, in comparison to a benchmark or predetermined threshold. In some embodiments, a microbiome may be strategically modulated based on acquired information. The devices and methods disclosed herein may also be used to culture and/or preserve a microbiome sample. In at least some embodiments, substantially the same sample may be, for example, both analyzed and cultured, in view of the sampling devices and methods disclosed herein.

“Microbiome” refers to a population, e.g., one or more microorganisms that live on a surface of a subject, e.g., in the gut, mouth, skin, and/or elsewhere in a subject. The population may have one or more beneficial functions and/or benefits, relevant to supporting the life of a subject.

In accordance with one or more embodiments, nonpathogenic bacteria may generally be autotrophic or heterotrophic. These bacteria may have beneficial properties, e.g., in connection with various cosmetic and therapeutic uses in accordance with one or more embodiments. Nonpathogenic bacteria of a microbiome sample may be associated with a predetermined criteria or reference. For example, nonpathogenic bacteria may be selected for ability to compete with pathogenic bacteria in the microbiome of a subject. In some embodiments, nonpathogenic bacteria may be selected for their ability to produce byproducts that inhibit growth or reproduction of pathogenic bacteria in the microbiome of a subject.

Certain bacteria, for example, including those commonly present in the microbiome of the lung, may inhibit the growth and reproduction of pathogenic bacteria therein. The most significant pathogenic bacteria of the lung include M. catarrhalis, H. influenzae, and S. pneumoniae. While not wishing to be bound by any particular theory, certain nonpathogenic bacteria from the respiratory microbiome may produce anti-inflammatory and antimicrobial particles, including, e.g., interleukin 10 (IL-10), FOXP3, and secretory immunoglobulin A (slgA), and induce a Th1 response, which inhibits pathological growth.

The nonpathogenic bacteria of this disclosure may be from a genus selected from the group consisting of Prevotella, Sphingomonas, Pseudomonas, Acinetobacter, Fusobacterium, Megasphaera, Veillonella, Staphylococcus, or Streptococcus, and combinations thereof. Nonpathogenic bacteria of such genera, for example, may inhibit the growth of pathogenic bacteria by colonizing the lung or delivering one or more product or byproduct to the lung.

Likewise, nonpathogenic bacteria of the nasal microbiome may inhibit the growth and reproduction of pathogenic bacteria therein. Specifically, S. pneumoniae and S. aureus, while commonly benign when present in the nasal passage microbiome, can sometimes become pathogenic and cause severe disease. It is not well understood what causes a pathogenic state of these bacteria, however, it is generally believed that colonization is a requirement for infection. While not wishing to be bound by any particular theory, certain nonpathogenic bacteria from the nasal microbiome, for example, C. accolens, may inhibit pneumococcal growth in the microbiome by releasing fatty acids that inhibit the pathogenic bacteria. It is believed that C. accolens releases lipase LipSI which hydrolyzes triacylglycerols, e.g., triolein, commonly found lining the nasal passages to release oleic acid, a fatty acid which inhibits pneumococcal growth.

The nonpathogenic bacteria of this disclosure may be from a genus selected from the group consisting of Staphylococcus, Corynebacterium, Propionibacterium, Rhodococcus, Microbacterium, or Streptococcus, and combinations thereof. Nonpathogenic bacteria of such genera, for example, may inhibit the growth of pathogenic bacteria by colonizing the nasal passages or delivering one or more product or byproduct to the nasal passages.

In some embodiments, the nonpathogenic bacteria may comprise one or more bacterium of the genus selected from the group consisting of Bacillus, Lactobacillus, Lactococcus, Streptomyces, Faecalibacterium, Bacteroides, or Bifidobacter and combinations thereof, e.g., L. rhamnosus, F. prausnitzii, and B. fragilis.

In some embodiments, nonpathogenic bacteria may be ammonia oxidizing microorganisms. The nonpathogenic bacteria, e.g., ammonia oxidizing microorganism may generate nitrite and/or nitric oxide from ammonia. Properties of autotrophic ammonia oxidizing bacteria (AOB), for example, are well described by Whitlock in U.S. Pat. No. 7,820,420, incorporated by reference herein, in its entirety for all purposes. In some embodiments, reference to ammonia oxidizing bacteria may be applicable to any ammonia oxidizing microorganisms (AOM), e.g., ammonia oxidizing bacteria (AOB) and ammonia oxidizing archaca (AOA).

Without wishing to be bound to any particular theory, due to the roles of nitrite and nitric oxide as important components of several physiological functions, such as vasodilation, inflammation and wound healing, these bacteria may have various beneficial properties for both healthy and immunopathological conditions. These bacteria are safe for use in humans because they are slow-growing, cannot grow on organic carbon sources, may be sensitive to soaps and antibiotics, and have never been associated with any disease or infection in animals or humans.

Ammonia oxidizing microorganisms generate coenzyme Q 8 (CoQ8) as a byproduct of the process by which they generate nitrite and nitric oxide. CoQ8 is a coenzyme Q having 8 carbons in its isoprenoid side chain. Without wishing to be bound to any particular theory, due to the role of coenzyme Q as an important component of several cell functions, such as mediating cell signaling and preventing cell death (anti-aging), these microorganisms' beneficial properties may further be enhanced by their specific ability to generate CoQ8.

The ammonia oxidizing bacteria of this disclosure may be from a genus selected from the group consisting of Nitrosomonas, Nitrosococcus, Nitrosospria, Nitrosocystis, Nitrosolobus, Nitrosovibrio, and combinations thereof. Examples of ammonia oxidizing bacteria include Nitrosomonas eutropha strains, e.g., D23 and C91 as discussed herein. D23 Nitrosomonas eutropha strain refers to the strain, designated AOB D23-100, deposited with the American Tissue Culture Collection (ATCC) (10801 University Blvd., Manassas, VA, USA) on Apr. 8, 2014 having accession number PTA-121157. The nucleic acid sequence(s), e.g., genome sequence, of accession number PTA-121157 are hereby incorporated herein by reference in their entireties for all purposes. “AOB D23-100” may also be referred to as D23 or B244 throughout this disclosure.

Examples of ammonia oxidizing archaea include archaea in the genera Methanobrevibacter, Methanosphaera, Methanosarcina, Nitroscaldus, Nitrosopumilus, and Nitrososphaera (e.g. Nitrososphaera viennensis, Nitrososphaera gargensis). Different phylotypes of archaea, e.g., methanogens and halphilic archaeon, may be included in the preparations disclosed herein. Examples of archaea further include archaea in the lineages of phyla Euryarchaeota (e.g. Methanosarcina), Crenarchaeota, Aigarchaeota, and Thaumarchaeota (e.g. Giganthauma karukerense, Giganthauma insulaporcus, Caldiarchaeum subterraneum, Cenarchaeum symbiosum).

This disclosure provides, inter alia, N. eutropha strain D23, a unique, e.g., optimized strain of ammonia oxidizing bacteria that can increase production of nitric oxide and nitric oxide precursors on a surface of a subject, e.g., a human subject. In certain embodiments, the N. eutropha strain comprises a nucleic acid sequence, e.g., a genome, that hybridizes to SEQ ID NO: 1 of International (PCT) Patent Application Publication No. WO2015160911 (International (PCT) Patent Application Serial No. PCT/US2015/025909 filed on Apr. 15, 2015, incorporated by reference herein, in its entirety for all purposes), or to the genome of the D23 strain deposited in the form of 25 vials with the ATCC patent depository on Apr. 8, 2014, designated AOB D23-100, under accession number PTA-121157, or their complements, under low stringency, medium stringency, high stringency, or very high stringency, or other hybridization condition.

The practice of the present invention may employ, unless otherwise indicated, conventional methods of immunology, molecular biology, and recombinant DNA techniques within the skill of the art. Such techniques are explained fully in the literature. See, e.g., Sambrook, et al. Molecular Cloning: A Laboratory Manual (Current Edition); and Current Protocols in Molecular Biology (F. M. Ausubel, et al. eds., current edition).

In accordance with one or more embodiments, a microbiome sampling device is disclosed. The device may generally include a capture substrate that is configured to obtain a microbiome sample from a subject. The capture substrate may generally be made of any material commonly known to those of skill in the art that is biocompatible and capable of contacting and retaining a microbiome sample. In some nonlimiting embodiments, the capture substrate may be made of a fiber material, such as a flocked material. In at least some nonlimiting embodiments, the capture substrate may be made of an absorbable material.

In accordance with one or more embodiments, the capture substrate may include various regions or segments, for example, a first region and a second region. Three, four, five or more regions are also contemplated. The capture substrate may be configured such that the various regions may be separable from each other, for example, after a microbiome sampling operation. Various regions or segments may acquire substantially the same microbiome sample from a subject during a sampling process. Each region may include a reagent suitable for a specific analytic method. For example, a first region of the capture substrate may include a first reagent suitable for a first analytic method, and a second region of the capture substrate may include a second reagent suitable for a second analytic method.

In accordance with one or more embodiments, the device may be configured to collect a sample from a target region of a subject. Any region of a subject may be targeted. For example, a microbiome sample may be obtained from the skin, face, neck, scalp, head, shoulder, arm, leg, underarm, torso, chest, feet, knee, ankle, buttocks, anus, digestive system, urogenital system, oral cavity, nasal cavity, eye, or ear of the subject.

As used herein, a “subject” may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, research, and agriculturally useful animals, for example, sheep, dog, cat, cow, pig, rat, mouse, rabbit, goat, among others.

In accordance with one or more embodiments, a subject may have a normal microbiome at a target region. In accordance with one or more other embodiments, a subject may have a disrupted microbiome at a target region.

The capture substrate may involve various configurations and may generally have a predetermined surface area. Various regions of capture substrate may be disposed on the same surface or on different surfaces. In accordance with one or more embodiments, the capture substrate may be in the form of a swab. In one embodiment, a single swab may include a first region and a second region adjacent to each other. The first region and second region may be separable. In an alternate embodiment, a first region of capture substrate may be a first swab, and a second region of capture substrate may be a second swab. The first and second swabs may be held adjacent together, for example, by a mechanical clamping element. The first and second swabs may share a handle. The first and second swabs may be separate or separable. The capture substrate may be disposed on the outer surface of the swab element and be configured to allow collection of a microbiome sample. The device may be configured such that upon movement across a surface, for example, in a linear, curved, or circular motion, the first and second portions of the capture substrate are brought into contact with a subject substantially simultaneously.

In accordance with one or more other embodiments, the capture substrate may be in the form of a roller. In one embodiment, a single roller may include a first region and a second region adjacent to each other. The first region and second region may be separable. In an alternate embodiment, a first region of capture substrate may be a first roller, and a second region of capture substrate may be a second roller. The first and second rollers may be held adjacent together, for example, by a mechanical clamping element. The first and second rollers may share a handle. The first and second rollers may be separate or separable. A roller element may have an outer surface and a transverse axis, with the roller element being configured to rotate about its transverse axis. The capture substrate may be disposed on the outer surface of the roller element and be configured to allow collection of a microbiome sample. The device may be configured such that upon movement across a surface perpendicular to the transverse axis, a first portion of the capture substrate is brought into contact with a subject, and upon further movement across the surface perpendicular to the transverse axis a second portion of the capture substrate is brought into contact with the subject.

Various other configurations of the device are within the scope of the invention so as to allow for microbiome sampling and, for example, subsequent analysis and culturing of a single microbiome sample through division or partitioning thereof.

In accordance with one or more embodiments, the capture substrate may be substantially sterile prior to contact with a subject. In some embodiments, the capture substrate may be substantially free of a preselected member of the microbiome, e.g. an AOM. In other embodiments, the capture substrate may have less than a threshold percentage of a preselected member of the microbiome, e.g., an AOM. In further embodiments, the capture substrate may have a predetermined level of a preselected member of the microbiome, e.g., an AOM.

In accordance with one or more embodiments, the device may be configured to apply any required amount of pressure or force to a target area of a subject to facilitate microbiome sample collection. In some embodiments, sampling by a first region of capture substrate may occur simultaneously with sampling by a second region of capture substrate. In other embodiments, sampling may occur in series. In at least some embodiments, sampling may involve contacting with a surface of a subject.

In accordance with one or more embodiments, the device may include a handle. The handle may be attached to the capture substrate. Then handle may have a grasping end and the capture substrate may be attached to a distal end of the handle relative to the grasping end. The handle itself may be configured to be separable into various portions. A first handle portion may be separable from a second handle portion. A first handle portion may be associated with a first region of capture substrate and a second handle portion may be associated with a second region of capture substrate. The handle may also be separable from the capture substrate. For example, the first handle portion may be separable from the first region of capture substrate, and the second handle portion may be separable from the second region of capture substrate.

In terms of separation between various regions of capture substrate and/or handle portions, the device may be configured with grooves, indents, perforations, or other structural features commonly known to those skilled in the art to promote such separation.

In accordance with one or more embodiments, the device may be configured for single use or multiple use. In some embodiments, the device may be configured such that after collection, a first loaded or used segment of capture substrate can be removed. After such removal, a second segment may be available for use.

In accordance with one or more embodiments, the capture substrate or region thereof may be configured to be received by a receptacle or container associated with a microbiome analytic method or for storage. In some embodiments, a first region of capture substrate may be received by a first receptacle and a second region of capture substrate may be received by a second receptacle.

In operation, the sampling device may be introduced to a subject. The capture substrate, i.e. swab or roller, may be contacted with a desired target area of a subject, i.e., skin, oral cavity, or nasal cavity. The contact may be sufficient to transfer microbiome sample from the target area of the subject to the capture substrate. In at least some embodiments, the contact may be sufficient to transfer microbiome sample from the target area of the subject to first and second regions of the capture substrate.

The contact may be performed such that movement across a surface may be performed, for example, in a linear, curved, or circular motion. The first and second portions of the capture substrate may be brought into contact with the same or substantially the same microbiome sample. The first and second portions of the capture substrate may be brought into contact with a subject substantially simultaneously.

The capture substrate containing microbiome sample may then undergo one or more analytic methods, culturing, or storage. In at least some embodiments, the capture substrate may be divided into its various regions, i.e. first region of capture substrate and second region of capture substrate. Such division may also involve separation of a handle attached to the capture substrate. Each portion of capture substrate may then be introduced to a discrete unit operation, i.e. analytic method, as discussed below.

In accordance with one or more embodiments, a single sampling operation may facilitate multiple analytic or other downstream methods. Each portion of capture substrate may be introduced to a receptacle associated with an analytic method. A first portion of capture substrate may undergo a first analytic method and a second portion of capture substrate may undergo a second analytic method. One or both portions of capture substrate may instead be used for culture or preserved for future use or reference. Detachment of the portion of capture substrate from its respective handle portion may be performed prior to or subsequent to introduction to a receptacle.

In accordance with one or more embodiments, a captured microbiome sample, i.e. a first portion of capture substrate, may be subjected to a first analytic method known to those of skill in the relevant art. Some analytic methods may involve nucleic acid sequencing, e.g., DNA or RNA sequencing. The nucleic acid sequencing may be performed with a next-generation sequencing (NGS) platform, multiplexed amplification operation with primers, nucleic acid manipulation, isolation, extraction, or purification of a nucleic acid, or contact with a restriction fragment. The first analytic method may involve culturing the sample, obtaining a snapshot of the sample, freezing, or otherwise preserving the sample.

In accordance with one or more embodiments, a captured microbiome sample, i.e. a second portion of capture substrate, may be subjected to a second analytic method. The second method may involve culturing the sample, obtaining a snapshot of the sample, freezing, or otherwise preserving the sample.

In accordance with one or more embodiments of the disclosed devices and methods, a single microbiome sample may be obtained and used in various analytic techniques and processes for precise, localized microbiome evaluation.

In accordance with one or more embodiments, a method of evaluating a microbiome sample from a subject may involve forming various reaction mixtures with microbiome samples. In some embodiments, a first reaction mixture may involve a first portion of capture substrate, and a second reaction mixture may involve a second portion of capture substrate. Each analytic method may use distinct reagents. Each analytic method may involve use of distinct buffer solutions. For example, a first reaction mixture may involve a first buffer solution and a second reaction mixture may involve a second buffer solution. The buffer solutions may or may not include a preservative.

In accordance with one or more embodiments, an analytic method may involve providing a value for a parameter related to a microbiome sample. A first value may relate to a first portion of capture substrate, and a second value may relate to a second portion of capture substrate. A first value may relate to a first analytic method, and a second value may relate to a second analytic method. For example, in some embodiments, one value may relate to a nucleic acid sequence of the microbiome sample. Another value may relate to a microbial colony forming unit (CFU) concentration.

In some embodiments, the values may be compared or correlated to predetermined or threshold values, such as may be associated with a profile of a normal or disrupted microbiome. In at least some embodiments, the values relating to various parameters of the microbiome sample(s) may be transmitted to a database. In certain embodiments, a microbiome of a subject may be modulated at a target area in response to a value for a parameter related to a microbiome sample of the subject.

In accordance with one or more embodiments, the device may be configured for direct to consumer applications. A subject may use the device to perform a microbiome sampling method. A loaded or used device may then be directed to a third party by the subject for analysis. In other embodiments, sampling and analysis may be performed by the same party.

In accordance with one or more embodiments, a microbiome sampling kit may include a device as described herein. The device may generally include a capture substrate. The device may also include a handle. The kit may further include instructions for using the device to sample microbiome of a subject based on methods as described herein and/or for evaluating a microbiome sample based on methods as described herein. The kit may further include receptacles and/or buffer solutions associated with various desired analytic methods. The kit may be substantially sterile. The kit may or may not be substantially free of preservative. The kit may include one or more other components to facilitate various analytic methods, such as but not limited to sources of lipid and or protein. In accordance with one or more embodiments, various components of a kit, e.g. the kit itself, each sampling device, capture substrate, and/or receptacle thereof may be barcoded to facilitate microbiome sampling, downstream processing, and/or analysis.

In accordance with one or more embodiments, a database may include values for one or more parameters associated with a microbiome of a subject. Such parameters may relate to one or more analytic methods associated with microbiome sampling. A processing system may correlate, compare, and/or map values in a database in connection with values from a sampling and/or analytic method. The database may relate to one or more subjects and may also include various information about the subject(s). The processing system may identify or characterize a microbiome of a subject, analyze the microbiome of the subject, and/or transmit information related to the microbiome of the subject. In at least some embodiments, a processing system may characterize a disease, disorder, or condition associated with a microbiome at a target area of a subject, for example, based on an analytic method and/or database of values as described herein. The microbiome of the subject may, in some embodiments, be modulated in response thereto, for example, to impart a cosmetic and/or therapeutic benefit to the subject.

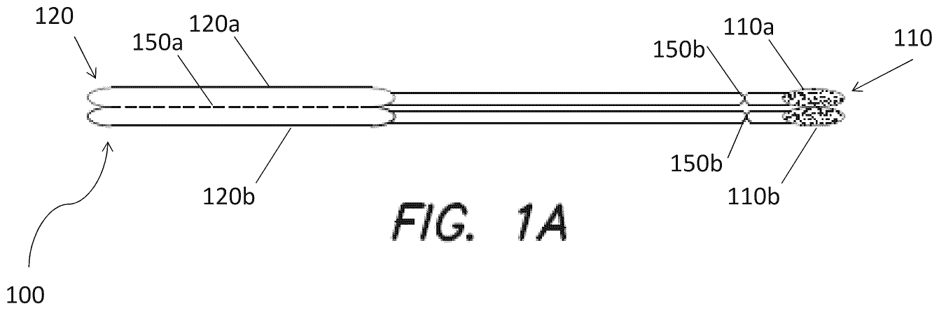

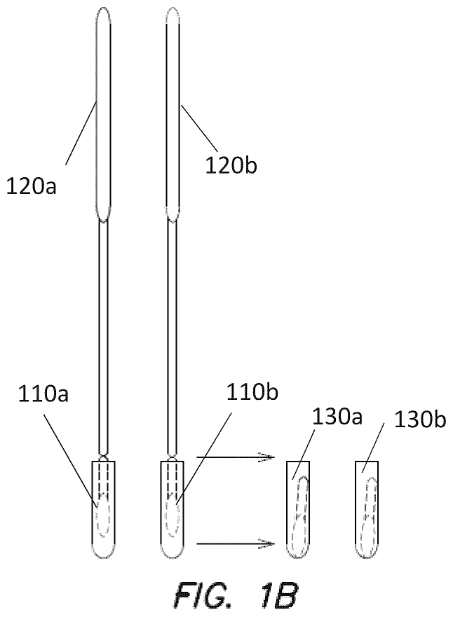

FIG. 1A provides a nonlimiting schematic of a device in accordance with one or more embodiments. Device 100 includes capture substrate 110 and handle 120. Capture substrate 110 includes first region 110a and second region 110b. Each capture substrate region 110a, 110b includes a swab. Handle 120 is separable into handle portion 120a and handle portion 120b via perforation feature 150a. Capture substrate 110 is likewise separable from handle 120 at perforation features 150b.

FIG. 1B provides a nonlimiting schematic of operation of the device of FIG. 1A. First region 110a and second region 110b of capture substrate have been introduced to a subject and have obtained a microbiome sample from a target site of the subject. The capture substrate now undergoes various analytic methods. Handle 120 is separated into handle portions 120a and 120b. First capture substrate region 110a is introduced to first receptacle 130a associated with a first analytic method. Second capture substrate region 110b is introduced to second receptacle 130b associated with a second analytic method. The receptacles may contain different buffer solutions. Subsequent to introduction, handle portions 120a, 120b may be separated from first and second capture regions 110a, 110b to facilitate further processing.

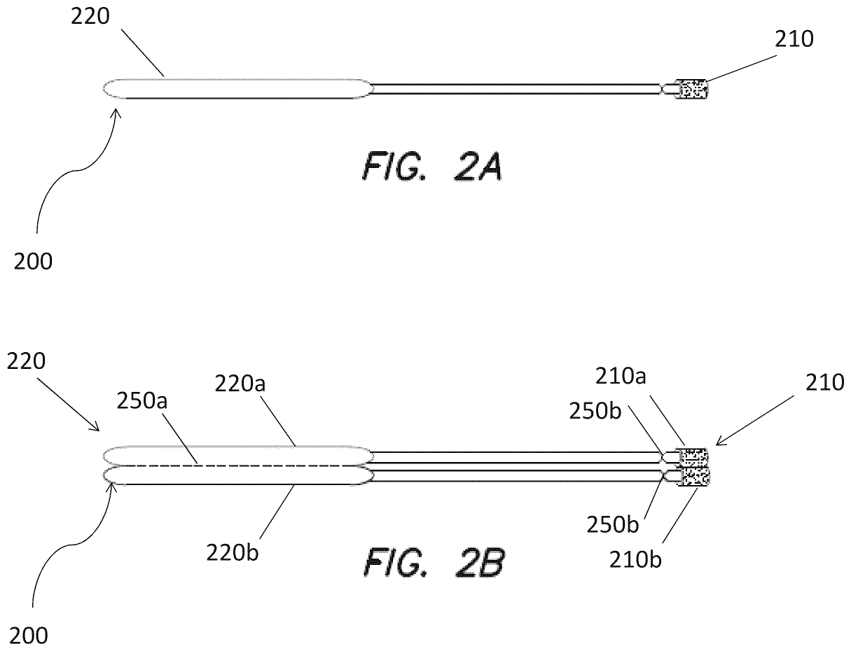

FIG. 2A provides a nonlimiting schematic of an alternative device in accordance with one or more embodiments. Device 200 includes capture substrate 210 and handle 220. Capture substrate 210 includes a roller element. FIG. 2B illustrates a rendition in which capture substrate 210 includes first region 210a and second region 210b. Each capture substrate region 210a, 210b includes a roller. Handle 220 is separable into handle portion 220a and handle portion 220b via perforation feature 250a. Capture substrate 210 is likewise separable from handle 220 at perforation features 250b. Device 200 including roller elements can likewise be used in operation analogous to that illustrated in FIG. 1B.

In accordance with one or more embodiments, the present disclosure provides for various methods of modulating a microbiome of a subject. These methods comprise administering to a subject microorganisms, for example, a preparation, composition, formulation, or product comprising nonpathogenic bacteria or archaea. In at least some embodiments, nonpathogenic microorganisms may therefore generally be restored to a microbiome of the subject. Such restoration may be in response to microbiome sampling and evaluation as described herein. In at least some embodiments, nonpathogenic microorganisms, e.g. ammonia oxidizing microorganisms, may comprise or consist essentially of beneficial bacteria or archaea.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

Certain embodiments are within the scope of the following claims.

1. A device for obtaining a microbiome sample from a subject, comprising:
a capture substrate, wherein the capture substrate is configured to contain:
a first region suitable for a first analytic method; and
a second region suitable for a second analytic method,
wherein the first region and the second region are configured to obtain microbiome sample from the subject.

2. A device for obtaining a microbiome sample from a subject, comprising:
a roller element having an outer surface and a transverse axis, the roller element configured to rotate about its transverse axis; and
a capture substrate disposed on the outer surface and configured to allow collection of a microbiome sample,
wherein the capture substrate is suitable for a first microbiome analytic method.

3. The device of any of the preceding claims, wherein the first and second regions obtain the same or substantially the same microbiome sample from the subject.

4. The device of any of the preceding claims, wherein the device is configured such that upon movement across a surface perpendicular to the transverse axis, a first portion of the capture substrate is brought into contact with the subject, and upon further movement across the surface perpendicular to the transverse axis a second portion of the capture substrate is brought into contact with the subject.

5. The device of any of the preceding claims, wherein, prior to contact with the subject:
(i) the capture substrate is substantially free of a preselected member of the microbiome, e.g. an AOM;
(ii) the capture substrate has less than a threshold percentage of a preselected member of the microbiome, e.g., an AOM; or
(iii) the capture substrate has a predetermined level of a preselected member of the microbiome, e.g., an AOM.

6. The device of any of the preceding claims, wherein the device comprises a plurality of segments of capture substrate.

7. The device of any of the preceding claims, wherein the device is configured such that after collection, a first loaded or used segment can be removed.

8. The device of any of the preceding claims, wherein the device is configured such that after removal of a first loaded or used segment, a second segment is available for use.

9. The device of any of the preceding claims, wherein the capture substrate comprises a predetermined surface area.

10. The device of any of the preceding claims, wherein the device is configured to apply a predetermined amount of pressure to a surface of the subject.

11. The device of any of the preceding claims, wherein the capture substrate comprises a reagent suitable for the first analytic method.

12. The device of any of the preceding claims, wherein the capture substrate is configured to contain:
a first region comprising a first reagent suitable for a first analytic method; and
a second region comprising a second reagent suitable for a second analytic method,
wherein the first region and the second region are configured to capture a microbiome sample from a subject.

13. The device of any of the preceding claims, further comprising a handle.

14. The device of any of the preceding claims, wherein the handle is configured to be separable into a first handle portion associated with the first region of capture substrate and a second handle portion associated with the second region of capture substrate.

15. The device of any of the preceding claims, wherein the handle is configured to be separated from the capture substrate.

16. The device of any of the preceding claims, wherein the first handle portion is separable from the second handle portion.

17. The device of any of the preceding claims, wherein the first handle portion is separable from the first region of capture substrate, and wherein the second handle portion is separable from the second region of capture substrate.

18. The device of any of the preceding claims, wherein the first region is disposed on a first surface, and the second region is disposed on a second surface.

19. The device of any of the preceding claims, wherein the first region and the second region are disposed on the same surface.

20. The device of any of the preceding claims, wherein the surface is configured, e.g., with a groove, indent, or perforation, to promote separation of the first region from the second region.

21. The device of any of the preceding claims, wherein the first and second regions sample microbiome simultaneously.

22. The device of any of the preceding claims, further comprising a second roller element.

23. The device of any of the preceding claims, wherein the capture substrate or region thereof comprises a swab or flock.

24. The device of any of the preceding claims, wherein the capture substrate is configured to be received by a receptacle associated with a microbiome analytic method.

25. A method of collecting a microbiome sample from a subject, comprising:
contacting the subject with a capture substrate,
wherein the capture substrate is configured to contain:
a first region suitable for a first analytic method; and
a second region suitable for a second analytic method,
wherein the first region and the second region are configured to sample microbiome from the subject, and
wherein the contacting is sufficient to transfer a microbiome sample to the capture substrate, thereby providing a microbiome sample from a subject.

26. A method of evaluating a microbiome sample from a subject, comprising:
a) providing a capture substrate configured to contain:
a first region suitable for a first analytic method; and
a second region suitable for a second analytic method,
wherein the first region and the second region are configured to capture microbiome sample from the subject, and
wherein the capture substrate has been contacted with the subject; and
b) forming one or both of:
(i) a first reaction mixture comprising microbiome sample from the first region; and
(ii) a second reaction mixture comprising microbiome sample from the second region.

27. The method of any of the preceding claims, wherein the contacting is sufficient to transfer microbiome sample from the subject to the first and second regions of the capture substrate.

28. The method of any of the preceding claims, wherein the contacting is sufficient to obtain the same or substantially the same microbiome sample from the subject on the first and second regions of the capture substrate.

29. The method of any of the preceding claims, wherein the contacting is sufficient to transfer microbiome sample from the subject to the first and second regions of the capture substrate substantially simultaneously.

30. The method of any of the preceding claims, further comprising separating the first region from the second region.

31. The method of any of the preceding claims, wherein the first region and/or the second region are contacted with the face, neck, scalp, head, shoulder, arm, leg, underarm, torso, chest, feet, knee, ankle, or buttocks of the subject.

32. The method of any of the preceding claims, wherein the capture substrate is configured to contain:
a first region comprising a first reagent suitable for a first analytic method; and
a second region comprising a second reagent suitable for a second analytic method,
wherein the first region and the second region are configured to capture a microbiome sample from a subject.

33. The method of any of the preceding claims, wherein the first region and the second region are configured to capture the same or substantially the same microbiome sample from the subject.

34. The method of any of the preceding claims, wherein the first region and the second region are configured to obtain the microbiome sample substantially simultaneously.

35. The method of any of the preceding claims, further comprising forming a first reaction mixture comprising the microbiome sample from the first region.

36. The method of any of the preceding claims, further comprising forming a second reaction mixture comprising the microbiome sample from the second region.

37. The method of any of the preceding claims, further comprising providing a value for a parameter related to the microbiome sample on the first region.

38. The method of any of the preceding claims, further comprising providing a value for a parameter related to a first analytic method, e.g., a nucleic acid sequence, e.g., DNA sequence or RNA sequence.

39. The method of any of the preceding claims, further comprising providing a value for a parameter related to the microbiome sample on the second region.

40. The method of any of the preceding claims, further comprising providing a value for a parameter related to the second analytic method, e.g., a microbial colony forming unit (CFU) concentration.

41. The method of any of the preceding claims, wherein the first analytic method comprises a manipulation of a nucleic acid.

42. The method of any of the preceding claims, wherein the first analytic method comprises isolation or purification of a nucleic acid.

43. The method of any of the preceding claims, wherein the first analytic method comprises sequencing a nucleic acid, e.g., DNA or RNA.

44. The method of any of the preceding claims, wherein the first analytic method comprises sequencing a nucleic acid with a NGS method.

45. The method of any of the preceding claims, wherein the first analytic method comprises contacting a nucleic acid with a restriction fragment.

46. The method of any of the preceding claims, wherein the second analytic method comprises culturing the microbiome sample.

47. The method of any of the preceding claims, wherein the second analytic method comprises obtaining a snapshot of the microbiome sample.

48. The method of any of the preceding claims, wherein the first or second analytic method involves preserving, e.g., freezing the microbiome sample.

49. The method of any of the preceding claims, wherein the capture substrate or region thereof comprises a roller.

50. The method of any of the preceding claims, wherein the capture substrate or region thereof comprises a swab or flock.

51. The method of any of the preceding claims, further comprising transmitting the value for a parameter related to the microbiome sample on the first and/or second region to a database.

52. The method of any of the preceding claims, further comprising correlating the value for a parameter related to the microbiome sample on the first and/or second region.

53. The method of any of the preceding claims, further comprising modulating a microbiome of the subject based on the value for a parameter related to the microbiome sample on the first and/or second region.

54. The method of any of the preceding claims, further comprising separating a handle associated with the capture substrate.

55. The method of any of the preceding claims, further comprising separating a handle from the capture substrate.

56. The method of any of the preceding claims, further comprising contacting the microbiome sample captured on the first region to a first reagent suitable for the first analytic method and contacting the microbiome sample captured on the second region to a second reagent suitable for the second analytic method.

57. The method of any of the preceding claims, further comprising introducing the capture substrate or region thereof to a buffer solution.

58. The method of any of the preceding claims, further comprising contacting the capture substrate with the skin, face, neck, scalp, head, shoulder, arm, leg, underarm, torso, chest, feet, knee, ankle, buttocks, anus, oral cavity, nasal cavity, eye, or ear of the subject.

59. A kit for evaluating a microbiome sample from a subject, comprising:
a collection device according to any of the preceding claims.

60. The kit of any of the preceding claims, further comprising instructions for performing any of the methods of providing a microbiome sample of any of the preceding claims.

61. The kit of any of the preceding claims, further comprising instructions for performing any of the methods of evaluating a microbiome sample of any of the preceding claims.

62. The kit of any of the preceding claims, further comprising a container for handling the capture substrate or region thereof.

63. The kit of any of the preceding claims, further comprising a source of one or more buffer solutions.

64. The kit of any of the preceding claims, wherein the kit and/or component(s) thereof are substantially sterile.

65. The kit of any of the preceding claims, wherein the kit and/or component(s) thereof are substantially free of preservatives.

66. The kit of any of the preceding claims, wherein the buffer solution comprises a preservative.

67. The kit of any of the preceding claims, further comprising a source of a protein or a lipid.

68. The kit of any of the preceding claims, wherein the kit is barcoded.

69. The kit of any of the preceding claims, wherein at least one component of the kit is barcoded.

70. A database comprising values for one or more parameters related to one or more microbiome analytic methods according to any of the preceding claims.
